(19) United States
(12) Patent Application Publication
(10) Pub. No.: US 2013/0061865 A1
(43) Pub. Date: Mar. 14, 2013

(54) SKIN TREATMENT COMPOSITION

(71) Applicants: Sameer Keshav Barne, Bangalore (IN); Kalpana Kamalakar Nayak, Bangalore (IN); Aravindakshan Perincheery, Bangalore (IN); Maya Treesa Saji, Bangalore (IN)

(72) Inventors: Sameer Keshav Barne, Bangalore (IN); Kalpana Kamalakar Nayak, Bangalore (IN); Aravindakshan Perincheery, Bangalore (IN); Maya Treesa Saji, Bangalore (IN)

(21) Appl. No.: 13/698,678

(22) PCT Filed: May 17, 2011
(86) PCT No.: PCT/EP2011/057953

§ 371 Date: Nov. 19, 2012

(30) Foreign Application Priority Data
May 31, 2010 (IN) 1650/MUM/2010

(51) Int. Cl. A61K 8/86; A61K 8/81; A61Q 17/00

(52) U.S. Cl. CPC A61K 8/86; A61Q 17/005; A61K 8/8147
USPC 132/200; 424/78.03

(57) ABSTRACT

The invention is in the field of skin hygiene, especially hand hygiene and/or hand soap compositions. It remains to be desired to prepare skin hygiene compositions having a high anti-microbial effect, with a low dosage of anti-microbial essential oils. It is therefore an object of the invention to provide a skin hygiene composition, having good anti-microbial properties, at low levels of essential oil. Surprisingly it has been found that composition comprising a low amount of essential oil and a polymer complex or mixture provides improved hygiene efficacy.

### FIELD OF THE INVENTION

The invention is in the field of skin hygiene, especially hand hygiene and/or hand soap compositions.

### BACKGROUND OF THE INVENTION

Skin hygiene is of high priority to present day consumers. Consumers all over the world use various kinds of skin hygiene compositions.

Skin generally contains several different micro-organisms in concentrations exceeding millions or even billions of colony forming units (cfu's) per square centimetre (cm²).

Many of these micro organisms are harmless, but there are also various pathogenic types or sub-species present, such as Escherichia coli, also referred to a E. coli, and Staphylococcus aureus, also referred to as S. aureus. Several other bacteria can be found in the skin flora, such as Staphylococcus epidermidis, also referred to as S. epidermidis, which is generally non-pathogenic, but is thought to be contributing to unpleasant body odour.

Therefore present day consumers appreciate skin care and cleansing products that have anti-microbial activity.

The most commonly known skin hygiene compositions predominantly consist of soap. Soap is a highly effective agent for killing bacteria. This is considered to be caused by its high alkalinity.

Various other skin hygiene materials have been proposed in the art. In recent years a number of publications have been made on the use of essential oils for anti-bacterial action.

In U.S. Pat. No. 5,965,518 essential oils are disclosed for use in fragrance compositions having antimicrobial activity.

In WO 01/70215, bactericidal composition comprising essential oils are disclosed for skin treatment and taught to reach even sub-dermal pathogens.

However, essential oils are relatively expensive ingredients. Additionally, essential oils are also known for their fragrances; using high amounts may cause a peculiar smell that is not always appreciated by the consumer.

Accordingly it remains to be desired to prepare skin hygiene compositions having a high anti-microbial effect, even with a low dosage of anti-microbial essential oils.

It is therefore an object of the invention to provide a skin hygiene composition, having good anti-microbial properties, at low levels of essential oil.

It is a further object of the invention to provide a composition that is effective against common skin and enteric bacteria, including both gram-positive and gram-negative bacteria.

Surprisingly it has been found that composition comprising a low amount of essential oil and a polymer complex or mixtures provides improved hygiene efficacy.

### SUMMARY OF THE INVENTION

Accordingly the present invention provides in a first aspect, a skin treatment composition comprising a polymer complex or mixture comprising a polymer A selected from the group of homopolymers and copolymers of carboxylic acid and derivatives, and a polymer B selected from the group of homopolymers and copolymers of alkylene oxides, vinyl pyrrolidone and/or their derivatives; and/or the group of homopolymers and copolymers of vinyl alcohol, saccharides, hydroxyalkyl cellulose and/or their derivates; and an essential oil selected from amyl salicylate, carvacrol, cymene, e.g. ρ-cymene, dihydroeugenol, eugenol, hexyl eugenol, hexyl salicylate, isoeugenol, methyl eugenol, methyl isoeugenol, methyl salicylate, tert butyl cresol, thymol, vanillin, cedrene, cineole, citral (including geranial and neral), citronellal, citronellol, eucalyptol (also known as 1,8 cineole) paradihydrolinalool, dihydromyrcenol (DH myrcenol), farnesol, geraniol, hexyl cinnamaldehyde, hydroxycitronallol, hydroxycitronellal, isocitral, limonene, preferably d-limonene, linalool, longifolene, menthol, nerol, nerolidiol, pinene, e.g. α-pinene, phellendrene, terpinene, e.g. α-terpinene and γ-terpinene, terpineol, e.g. γ-terpineol and terpin-4-ol, and tetrahydromyrcenol (THM), and wherein Polymers A and B are not the same

In a second aspect the invention provides a method for providing an anti-microbial effect to skin comprising the steps of applying a composition according to the invention to the skin, and waiting for at least 5 seconds.

In a third aspect the invention provides the use of a combination of a polymer complex or mixture comprising polymer A selected from the group of homopolymers and copolymers of carboxylic acid and derivatives, and a polymer B selected from the group of homopolymers and copolymers of alkylene oxides, vinyl pyrrolidone and/or their derivatives; and/or the group of homopolymers and copolymers of vinyl alcohol, saccharides, hydroxyalkyl cellulose and/or their derivates; and an essential oil, for providing an anti-microbial effect on skin.

By anti-microbial effect is meant being able to kill bacteria by at least 2 log (a factor 100) within 1 minute under standard test conditions (e.g. ASTM E2149-01) in-vitro.

By skin treatment composition is meant any composition for application onto skin. By skin is meant any keratinous substrate on the external surface of the body, including but not limited to, hands, face, underarm, hair and scalp.

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

### DETAILED DESCRIPTION OF THE INVENTION

The composition according to the invention thus comprises a polymer complex or mixture and an essential oil.

Polymer Complex or Mixture

The polymer complex according to the invention comprises a polymer A selected from the group of homopolymers and copolymers of carboxylic acid and derivatives, and a polymer B selected from the group of homopolymers and copolymers of alkylene oxides, vinyl pyrrolidone and/or their derivatives;

and/or the group of homopolymers and copolymers of vinyl alcohol, saccharides, hydroxyalkyl cellulose and/or their derivates;

The composition according to the invention comprises a polymer A and a polymer B. Polymers A and B are typically selected such that they form a complex due to the formation of hydrogen bonds.

The polymers may be homo polymers or co polymers, wherein by copolymer of monomer X is meant any polymer that contains the monomer X and at least one further monomer.

Polymers A and B are preferably present in the composition in a ratio of between 1:5 and 5:1, more preferably between 1:2 and 2:1.

Polymer A

According to the present invention, polymer A is a polymer selected from the group of homopolymers and copolymers of carboxylic acid and derivatives. Polymer A has a plurality of carboxyl groups. The polymer A has a molecular mass preferably from 300 to 10⁹ D (Dalton, also referred to as atomic mass units, amu). The polymer A is selected from the class consisting of homopolymers or copolymers of carboxylic polymers, including natural synthetic and semi-synthetic polymers in this class.

Some non-limiting examples of polymer A according to the present invention include:

(a) homopolymers of a carboxylic acid, including but not limited to polycarboxylic acid such as polyacrylic acid, polymaleic acid or copolymers of acrylic and maleic acid.

(b) polysaccharides comprising carboxyl groups. Such polysaccharides may include (but are not limited to) starch, cellulose, sodium alginate, natural gums, and their modified materials such as sodium carboxymethyl cellulose, hydroxyethyl cellulose.

Homopolymers or copolymers of carboxylic acid have a molecular mass of preferably from 2×10³ to 10⁷ D more preferably from 5×10⁴ to 10⁶ D and most preferably from 9×10⁴ to 5×10⁵ D.

If the polymers are in particulate form, the particle size is preferably less than 200 μm, preferably less than 100 μm, more preferably less than 50 μm still more preferably less than 10 μm, or even less than 5 μm.

The homopolymers or copolymers of polysaccharide have a molecular mass of preferably from 10³ to 10⁹ D, more preferably from 10⁴ to 10⁹ D and most preferably from 10⁵ to 10⁹ D.

Polymer A is preferably at least partially neutralised in the Sodium (Na⁺) form, preferably at least 10% w of polymer A is neutralised, more preferably at least 20%, still more preferably at least 50%.

Polymer A may be synthetic, semi-synthetic or natural. However, synthetic or semi-synthetic polymers are preferred.

Polymer A is preferably water soluble or water dispersible, most preferably polymer A is water soluble.

It is preferred that the polymer A is selected from a class consisting of homopolymers or copolymers of carboxylic acid.

The homopolymers or copolymers of carboxylic acid are preferably a polyacrylic acid or a copolymer thereof. Examples include SOKALAN® PA (BASF) and CARBOPOL® (Lubrizol).

The concentration of polymer A in the composition according to the invention is preferably between 0.001 and 25% by weight, more preferably at least 0.002%, or even at least 0.005%, but preferably not more than 15%, more preferably less than 5%, still more preferably less than 1%, even more preferably less than 0.5%, even less than 0.1%, or even less than 0.05% by weight of the composition.

Polymer B

According to the present invention, polymer B has a monomeric unit comprising a group that can form hydrogen bonds with the carboxyl groups of polymer A.

Accordingly, polymer B is selected from the group of homopolymers and copolymers of alkylene oxides, vinyl pyrrolidone and/or their derivatives; and/or the group of homopolymers and copolymers of vinyl alcohol, saccharides, hydroxyalkyl cellulose and/or their derivates.

The group of homopolymers and copolymers of vinyl alcohol, saccharides, hydroxyalkyl cellulose and/or their derivates, is generally not water soluble. In order to obtain the benefit of this group of polymers the particle size is set such that the particles are easily dispersible in water or and aqueous solution (i.e. a wash or rinse liquor). If the polymers are in particulate form, the particle size is preferably less than 200 μm, more preferably less than 100 μm, even more preferably less than 50 μm still more preferably less than 10 μm, or even less than 5 μm.

Polymers and homopolymers of carboxylic acid and/or sacchharides and/or polyalkylene glycol/ether qualify to be selected both as polymer A or polymer B, as they comprise hydroxyl or carboxyl group and either a carbonyl or an ether group. However, according to a preferred embodiment, polymer A and polymer B are not the same. It is particularly preferred that the polymers A and B are selected from different classes of polymers. Without wishing to be limited by theory, it is believed that the two polymers A and B, when dissolved in water, form a complex with a solubility lower than each of the polymers A and B, which helps in enhanced deposition and other benefits.

Polymer B preferably has a molecular mass from 10³ to 10⁹ D.

Homopolymers or copolymers of vinyl pyrrolidone or vinyl alcohol preferably have a molecular mass of between 10³ and 10⁷ D, more preferably from 10⁴ to 10⁶ D and most preferably from 30,000 to 500,000 D. Commercially available polyvinyl pyrrolidone can be used, one example of which is LUVISKOL® (BASF).

Homopolymers or copolymers of poly alkylene oxide preferably have a molecular mass greater than 2×10⁴ D. The molecular mass is preferably from 2×10⁴ to 10⁶ D, more preferably from 3×10⁴ to 5×10⁵ D and most preferably from 5×10⁴ to 2×10⁵ D.

Homopolymers or copolymers of saccharide preferably have a molecular mass of preferably from 10³ to 10⁹ D, more preferably from 10⁴ to 10⁹ D and most preferably from 10⁵ to 10⁹ D. Any commercially available poly alkylene oxide, for example POLYOX® (Dow Chemical Co) can be used according to the present invention.

Polymer B may be synthetic, semi-synthetic or natural. However, synthetic or semi-synthetic polymers are preferred.

According to a preferred embodiment, the polymer B is water soluble.

It is particularly preferred that the polymer B is selected from a class consisting of homopolymers or copolymers of vinyl pyrrolidone or alkylene oxide.

The concentration of polymer B in the composition according to the invention is preferably between 0.001 and 20% by weight, more preferably at least 0.002%, or even at least 0.005%, but preferably not more than 10%, more preferably less than 5%, still more preferably less than 1%, even more preferably less than 0.5%, even less than 0.1%, or even less than 0.05% by weight of the composition.

Some examples of combinations of polymer A and polymer B, which are particularly preferred, are given below.

TABLE 1
Preferred combination of the polymers

| Polymer A | Polymer B |
|---|---|
| Polyacrylic acid (PAA) | Poly vinyl pyrrolidone |
| | (PVP) |
| Polyacrylic acid (PAA) | Polyethylene Oxide(PEO) |
| Polyacrylic acid (PAA) | Polyethylene Glycol (PEG) |
| Polyacrylic acid (PAA) | Poly vinyl alcohol (PVA) |
| Poly vinyl alcohol (PVA) | Polyethylene Oxide (PEO) |
| Sodium carboxymethyl cellulose | Polyethylene Oxide (PEO) |
| (SCMC) | |
| Polyacrylic acid(PAA) | Hydroxyethyl cellulose |
| Starch-graft-polymethacrylic acid | Polyethylene Oxide |
| Starch-graft-polymethacrylic acid | Polyvinyl pyrrolidone |
| Pluronic-g-Polyacrylic acid | Polyethylene Oxide |
| Pluronic-g-Polyacrylic acid | Polyvinyl pyrrolidone |
| Sodium carboxymethyl cellulose | Hydroxyethyl cellulose |
| Sodium carboxymethyl cellulose | Polyvinyl alcohol |

The most preferred combinations of the polymers are PAA-PVP, PAA-PEO, PAA-PEG, Starch-graft-polymethacrylic acid-Polyethylene Oxide.

Essential Oil

Essential oils are typically concentrated, hydrophobic liquid containing volatile aroma compounds from plants. Essential oils may also be obtained though synthetic or semi-synthetic routes. Essential oils are also known as volatile, ethereal oils or aetherolea. An oil is “essential” in the sense that it carries a distinctive scent, or essence, of the plant. Essential oils do not, as a group, need to have any specific chemical properties in common, beyond conveying characteristic fragrances.

Essential oils are generally extracted by distillation. Other processes include expression, or solvent extraction. They are used in perfumes, cosmetics, soap and other products, for flavouring food and drink, and for scenting incense and household cleaning products.

Examples of aromatic essential oils suitable for use in the present invention include amyl salicylate, carvacrol, cymene, e.g. ρ-cymene, dihydroeugenol, eugenol, hexyl eugenol, hexyl salicylate, isoeugenol, methyl eugenol, methyl isoeugenol, methyl salicylate, tert butyl cresol, thymol, and vanillin. Examples of non-aromatic essential oils of terpenoid compounds include cedrene, cineole, citral (including geranial and neral), citronellal, citronellol, eucalyptol (also known as 1,8 cineole) paradihydrolinalool, dihydromyrcenol (DH myrcenol), farnesol, geraniol, hexyl cinnamaldehyde, hydroxycitronallol, hydroxycitronellal, isocitral, limonene, preferably d-limonene, linalool, longifolene, menthol, nerol, nerolidiol, pinene, e.g. α-pinene, phellendrene, terpinene, e.g. α-terpinene and γ-terpinene, terpineol, e.g. γ-terpineol and terpin-4-ol, and tetrahydromyrcenol (THM).

The most preferred essential oils in the context of the present invention are thymol, terpineol, eugenol, or mixture thereof.

The essential oil is preferably present in the composition in a concentration of between 0.001 and 10% by weight of the composition, but preferably at least 0.002%, or even at least 0.005% by weight of the composition, while preferably not more than 5%, more preferably not more than 1%, still more preferably not more than 0.5%, or even not more than 0.1% by weight of the concentration.

It is preferred that the composition comprises a second essential oil, wherein the essential oils are even more preferably selected from any combination of a thymol, a terpineol and/or a eugenol.

It is even more preferred that the composition comprises three essential oils, wherein the essential oils are still more preferably selected from a combination of a thymol, a terpineol and a eugenol.

When more than one essential oil is present in the composition, the above mentioned concentrations may be considered to be the concentrations of the combined essential oils, but preferably relate to each of the individual essential oils.

Compositions

The compositions according to the invention may be applied in various skin care and cleansing products, including but not limited to hand soap, hand hygiene, deodorants, face wash, body wash and even shampoo and hair conditioner products. It is preferred that the compositions are applied to the skin neat, while the skin may be wet or dry at the time of application.

It is preferred that the contact time of the product with the skin before rinsing is at least 5 seconds, more preferably at least 10 seconds, still more preferably at least 15 seconds, or even at least 20 seconds.

Stay on compositions, such as deodorants, skin hygiene compositions, skin care compositions may even stay for a longer period of time, preferably at least 1 minute, more preferably at least 15 minutes, still more preferably at least 1 hour, still more preferably at least 2 hours, or even more than 5 hours.

The pH of the compositions is preferably neutral or mildly acidic, more preferably between pH 2 and 9, still more preferably at least pH 3, while more preferably less than pH 8, still more preferably less than pH 7, or even less than pH 6.

Method

Consequently there is provided a method for providing an anti-microbial effect to skin comprising the steps of applying a composition according to the invention to the skin, waiting for at least 5 seconds, preferably at least 15 seconds, more preferably at least 1 minute, or even more than 2 minutes.

For hand/skin hygiene applications, skin care applications and deodorant applications the composition is preferably left on the skin after application without rinsing, but may be wiped of after the indicated time.

For hand soap, face and body wash and shampoo and hair conditioner applications, the skin is preferably rinsed after application and after the indicated time.

The use of the combination of the polymer complex or mixture according to the invention and the essential oil, is for providing an anti-microbial effect on skin, and preferably excludes therapeutic applications.

### EXAMPLES

The invention will now be illustrated by means of the following non-limiting examples.

The protocol used for testing in-vitro is based on standard test method ASTM E2149-01, wherein working cultures of individual bacterial species (S. epidermidis ATCC 12228 or E. coli ATCC 10536 as indicated below) were added to the test samples; and were given a 15 second contact time. After 15 seconds, the samples were neutralized and serially diluted in a neutralizer. The viable count is determined by agar pour plating. Activity is assessed by comparing the size of the population of untreated with that of treated specimens.

Unless stated otherwise this test method is used in the examples below.

### Example 1

### Anti-Microbial Efficacy Test (In Vitro) Against E. coli

Test compositions and bacterial kill results are given in the table below:

| | Blank | Comp A | Comp B | Comp C | Ex 1 | Ex 2 |
|---|---|---|---|---|---|---|
| | (% w) | (% w) | (% w) | (% w) | (% w) | (% w) |
| E. coli | 320 × 10⁶ | 320 × 10⁶ | 320 × 10⁶ | 320 × 10⁶ | 320 × 10⁶ | 320 × 10⁶ |
| (start, | | | | | | |
| cfu/ml) | | | | | | |
| Polymer | | 0.025 | | | 0.025 | 0.025 |
| complex ¹⁾ | | | | | | |
| Terpineol | | | 0.125 | 0.25 | 0.125 | 0.25 |
| Saline | balance | balance | balance | balance | balance | balance |
| solution ²⁾ | | | | | | |
| Kill | 320 × 10⁶ | 300 × 10⁶ | 280 × 10⁶ | 4 × 10⁶ | 1 × 10⁶ | 0 |
| (cfu/ml | | | | | | |
| remaining) | | | | | | |
| Kill (log | 0.0 | 0.0 | 0.1 | 1.9 | 2.2 | 8.5 |
| reduction) | | | | | | |

¹⁾ The polymer complex comprised PAA (poly acrylic acid; Mw 100,000 D, ex Sigma-Aldrich) and PEO (poly ethylene oxide; Mw 100,000 D, ex Sigma-Aldrich), in a ratio of 1.5:1 in a total amount as given in the table.
²⁾ The saline solution comprised 0.1% NaCl and Citric acid to a pH of 3.6.

The table above shows that the compositions according to the invention provide substantially better anti-microbial activity than the sum of the activities of each of the individual components.

### Example 2

### Anti-Microbial Efficacy Test (In Vitro) Against S. epidermidis

Test compositions and bacterial kill results are given in the table below:

| | Blank | Comp A | Comp B | Comp C | Ex 1 | Ex 2 |
|---|---|---|---|---|---|---|
| | (% w) | (% w) | (% w) | (% w) | (% w) | (% w) |
| S. epidermidis | 11.5 × 10⁶ | 11.5 × 10⁶ | 11.5 × 10⁶ | 11.5 × 10⁶ | 11.5 × 10⁶ | 11.5 × 10⁶ |
| (start, | | | | | | |
| cfu/ml) | | | | | | |
| Polymer | | 0.025 | | | 0.025 | 0.025 |
| complex ¹⁾ | | | | | | |
| Terpineol | | | 0.125 | 0.25 | 0.125 | 0.25 |
| Saline | balance | Balance | balance | balance | Balance | Balance |
| solution ²⁾ | | | | | | |
| Kill | 11.5 × 10⁶ | 10.5 × 10⁶ | 9.75 × 10⁶ | 7.75 × 10⁶ | 0.81 × 10⁶ | 0 |
| (cfu/ml | | | | | | |
| remaining) | | | | | | |
| Kill (log | 0.0 | 0.1 | 0.1 | 0.2 | 1.2 | 7.1 |
| reduction) | | | | | | |

¹⁾ The polymer complex comprised PAA (poly acrylic acid; Mw 100,000 D, ex Sigma-Aldrich) and PEO (poly ethylene oxide; Mw 100,000 D, ex Sigma-Aldrich), in a ratio of 1.5:1 in a total amount as given in the table.
²⁾ The saline solution comprised 0.1% NaCl and Citric acid to a pH of 3.6.

The table above shows that the compositions according to the invention provide substantially better anti-microbial activity than the sum of the activities of each of the individual components.

### Example 3

### Anti-Microbial Efficacy Test (In Vitro) Against E. coli

Test compositions and bacterial kill results are given in the table below:

| | Blank | Comp D | Comp E | Ex 3 |
|---|---|---|---|---|
| | (% w) | (% w) | (% w) | (% w) |
| E. coli (start, | 32 × 10⁶ | 32 × 10⁶ | 32 × 10⁶ | 32 × 10⁶ |
| cfu/ml) | | | | |
| Polymer complex¹⁾ | | 0.025 | | 0.025 |
| Eugenol | | | 0.1 | 0.1 |
| Saline solution²⁾ | Balance | Balance | Balance | balance |
| Kill (cfu/ml | 32 × 10⁶ | 27 × 10⁶ | 20 × 10⁶ | 110 |
| remaining) | | | | |
| Kill (log | 0.0 | 0.1 | 0.2 | 5.5 |
| reduction) | | | | |

¹⁾The polymer complex comprised PVA (poly vinyl alcohol, 89% degree of hydrolysis and 125,000 D, from SD fine Chem) and PEO (poly ethylene oxide; Mw 100,000 D, ex Sigma-Aldrich), in a ratio of 1.5:1 in a total amount as given in the table.
²⁾The saline solution comprised 0.1% NaCl and Citric acid to a pH of 3.6.

The table above shows that the composition according to the invention (Ex 3, example composition 3) provides substantially better anti-microbial activity than the sum of the activities of each of the individual components.

### Example 4

### Anti-Microbial Efficacy Test (In Vitro) Against E. coli

Test compositions and bacterial kill results are given in the table below:

| | Blank | Comp D | Comp N | Comp O | Ex 4a | Ex 4b |
|---|---|---|---|---|---|---|
| | (% w) | (% w) | (% w) | (% w) | (% w) | (% w) |
| E. coli | 32 × 10⁶ | 32 × 10⁶ | 32 × 10⁶ | 32 × 10⁶ | 32 × 10⁶ | 32 × 10⁶ |
| (start, | | | | | | |
| cfu/ml) | | | | | | |
| Polymer | | 0.025 | | | 0.025 | 0.025 |
| complex ¹⁾ | | | | | | |
| Thymol | | | 0.05 | 0.1 | 0.05 | 0.1 |
| Saline | Balance | Balance | Balance | Balance | balance | Balance |
| solution ²⁾ | | | | | | |
| Kill | 32 × 10⁶ | 27 × 10⁶ | 30 × 10⁶ | 20 × 10⁶ | 11 | 8 |
| (cfu/ml | | | | | | |
| remaining) | | | | | | |
| Kill (log | 0.0 | 0.1 | 0.0 | 0.1 | 7.1 | 7.1 |
| reduction) | | | | | | |

¹⁾ The polymer complex comprised PVA (poly vinyl alcohol, 89 % degree of hydrolysis and 125,000 D, from SD fine Chem) and PEO (poly ethylene oxide; Mw 100,000 D, ex Sigma-Aldrich), in a ratio of 1.5:1 in a total amount as given in the table.
²⁾ The saline solution comprised 0.1% NaCl and Citric acid to a pH of 3.6.

The table above shows that the compositions according to the invention provide substantially better anti-microbial activity than the sum of the activities of each of the individual components.

### Example 5

### Anti-Microbial Efficacy Test (In Vitro) Against E. coli with 2 Essential Oils

Test compositions and bacterial kill results are given in the table below:

| | Blank | Comp F | Comp G | Ex 5 | Ex 6 | Ex 7 |
|---|---|---|---|---|---|---|
| | (% w) | (% w) | (% w) | (% w) | (% w) | (% w) |
| E. coli | 30 × 10⁶ | 30 × 10⁶ | 30 × 10⁶ | 30 × 10⁶ | 30 × 10⁶ | 30 × 10⁶ |
| (start, | | | | | | |
| cfu/ml) | | | | | | |
| Polymer | | 0.025 | | 0.025 | 0.025 | 0.025 |
| complex ¹⁾ | | | | | | |
| Thymol | | | 0.025 | 0.0125 | 0.02 | 0.025 |
| Terpineol | | | 0.062 | 0.031 | 0.05 | 0.062 |
| Saline | balance | balance | balance | Balance | balance | balance |
| solution ²⁾ | | | | | | |
| Kill | 30 × 10⁶ | 29 × 10⁶ | 5.4 × 10⁶ | 2.2 × 10⁶ | 0.63 × 10⁶ | 0 |
| (cfu/ml | | | | | | |
| remaining) | | | | | | |
| Kill (log | 0.0 | 0.1 | 0.8 | 1.2 | 1.7 | 7.5 |
| reduction) | | | | | | |

¹⁾ The polymer complex comprised PAA (poly acrylic acid; Mw 100,000 D, ex Sigma-Aldrich) and PEO (poly ethylene oxide; Mw 100,000 D, ex Sigma-Aldrich), in a ratio of 1.5:1 in a total amount as given in the table.
²⁾ The saline solution comprised 0.1% NaCl and Citric acid to a pH of 3.6.

The table above shows that the compositions according to the invention provide substantially better anti-microbial activity than the sum of the activities of each of the individual components.

### Example 6

### Anti-Microbial Efficacy Test (In Vitro) Against E. coli—Effect of Polymer Concentration

Test compositions and bacterial kill results are given in the table below:

| | Blank | Comp F | Comp G | Ex 7 | Ex 8 | Ex 9 |
|---|---|---|---|---|---|---|
| | (% w) | (% w) | (% w) | (% w) | (% w) | (% w) |
| E. coli | 30 × 10⁶ | 30 × 10⁶ | 30 × 10⁶ | 30 × 10⁶ | 30 × 10⁶ | 30 × 10⁶ |
| (start, | | | | | | |
| cfu/ml) | | | | | | |
| Polymer | | 0.025 | | 0.025 | 0.0125 | 0.0125 |
| complex ¹⁾ | | | | | | |
| Thymol | | | 0.025 | 0.025 | 0.0125 | 0.025 |
| Terpineol | | | 0.062 | 0.062 | 0.031 | 0.062 |
| Saline | balance | balance | balance | balance | balance | balance |
| solution ²⁾ | | | | | | |
| Kill | 30 × 10⁶ | 30 × 10⁶ | 3.35 × 10⁶ | 0 | 1.7 × 10⁶ | 0.23 × 10⁶ |
| (cfu/ml | | | | | | |
| remaining) | | | | | | |
| Kill (log | 0.0 | 0.0 | 0.7 | 7.5 | 1.3 | 2.1 |
| reduction) | | | | | | |

¹⁾ The polymer complex comprised PAA (poly acrylic acid; Mw 100,000 D, ex Sigma-Aldrich) and PEO (poly ethylene oxide; Mw 100,000 D, ex Sigma-Aldrich), in a ratio of 1.5:1 in a total amount as given in the table.
²⁾ The saline solution comprised 0.1% NaCl and Citric acid to a pH of 3.6.

The table above shows that the compositions according to the invention provide substantially better anti-microbial activity than the sum of the activities of each of the individual components, even when the concentrations are reduced.

### Example 7

### Anti-Microbial Efficacy Test (In Vitro) Against E. coli—Effect of Individual Polymers

Test compositions and bacterial kill results are given in the table below:

| | Blank | Comp D | Comp G | Comp I | Comp J | Ex 10 |
|---|---|---|---|---|---|---|
| | (% w) | (% w) | (% w) | (% w) | (% w) | (% w) |
| E. coli | 32 × 10⁶ | 32 × 10⁶ | 32 × 10⁶ | 32 × 10⁶ | 32 × 10⁶ | 32 × 10⁶ |
| (start, | | | | | | |
| cfu/ml) | | | | | | |
| PVA ¹⁾ | | 0.015 | | 0.015 | | 0.015 |
| PEO ¹⁾ | | 0.01 | | | 0.01 | 0.01 |
| Thymol | | | 0.025 | 0.025 | 0.025 | 0.025 |
| Terpineol | | | 0.062 | 0.062 | 0.062 | 0.062 |
| Saline | balance | balance | balance | Balance | balance | balance |
| solution ²⁾ | | | | | | |
| Kill | 32 × 10⁶ | 27 × 10⁶ | 1.7 × 10⁶ | 0.018 × 10⁶ | 0.17 × 10⁶ | 0 |
| (cfu/ml | | | | | | |
| remaining) | | | | | | |
| Kill (log | 0.0 | 0.1 | 0.3 | 3.2 | 2.3 | 7.5 |
| reduction) | | | | | | |

¹⁾ The polymer complex comprised PVA (poly vinyl alcohol, degree of hydrolysis 89%, molecular weight 125,000 D and Ex SDFine Chem) and PEO (poly ethylene oxide; Mw 100,000 D, ex Sigma-Aldrich), in a ratio of 1.5:1 in a total amount as given in the table, or its individual components.
²⁾ The saline solution comprised 0.1% NaCl and Citric acid to a pH of 3.6.

The table above shows that the compositions according to the invention provide substantially better anti-microbial activity than the sum of the activities of each of the individual components, even when the concentrations are reduced.

### Example 8

### Anti-Microbial Efficacy Test (In Vitro) Against E. coli—with Three Essential Oils

Test compositions and bacterial kill results are given in the table below:

| | Blank | Comp F | Comp N | Ex 11 | Ex 12 | Ex 13 |
|---|---|---|---|---|---|---|
| | (% w) | (% w) | (% w) | (% w) | (% w) | (% w) |
| E. coli | 24 × 10⁶ | 24 × 10⁶ | 24 × 10⁶ | 24 × 10⁶ | 24 × 10⁶ | 24 × 10⁶ |
| (start, | | | | | | |
| cfu/ml) | | | | | | |
| PAA ¹⁾ | | 0.015 | | 0.015 | 0.015 | 0.015 |
| PEO ¹⁾ | | 0.01 | | 0.01 | 0.01 | 0.01 |
| Thymol | | | 0.025 | 0.0067 | 0.0125 | 0.025 |
| Terpineol | | | 0.062 | 0.016 | 0.031 | 0.062 |
| Eugenol | | | 0.045 | 0.023 | 0.045 | 0.045 |
| Saline | balance | balance | Balance | balance | balance | Balance |
| solution ²⁾ | | | | | | |
| Kill | 24 × 10⁶ | 17 × 10⁶ | 17 × 10⁶ | 8.1 × 10⁶ | 0 | 0 |
| (cfu/ml | | | | | | |
| remaining) | | | | | | |
| Kill (log | 0.0 | 0.2 | 0.2 | 0.5 | 7.4 | 7.4 |
| reduction) | | | | | | |

¹⁾ The polymer complex comprised PAA (poly acrylic acid; Mw 100,000 D, ex Sigma-Aldrich) and PE) (poly ethylene oxide; Mw 100,000 D, ex Sigma-Aldrich), in a total amount as given in the table, or its individual components.
²⁾ The saline solution comprised 0.1% NaCl and Citric acid to a pH of 3.5.

The table above demonstrates that the best results are obtained with very low concentrations of 3 essential oils and the polymer complex according to the invention.

### Example 9

### A Typical Hand Sanitizer According the Invention

A typical hand sanitizer composition according to the invention is given in the table below.

| Ingredients | % wt/wt |
|---|---|
| Phenoxy ethanol | 0.2 |
| Disodium EDTA | 0.05 |
| Methyl paraben | 0.2 |
| Terpineol | 0.05 |
| Thymol | 0.025 |
| Eugenol | 0.005 |
| Citric acid monohydrate | 0.2 |
| Poly ethylene oxide | 0.01 |
| Carbopol ETD 2020 (poly acrylic acid) | 0.015 |
| DM water | rest |
| Total | 100 |

The composition given above provides long lasting hygiene when applied to skin.

### Example 10

### Hand Soap Composition

| Ingredients | % wt/wt |
|---|---|
| SODIUM LAURETH SULFATE 1EO 70% | 10 |
| Cocoamidopropyl betaine | 10 |
| GLYCERIN | 2 |
| ISOPROPYL PALMITATE | 0.25 |
| Poly acrylic acid (PAA) | 0.03 |
| Poly ethylene oxide (PEO) | 0.02 |
| Citric acid mono hydrate | 0.50 |
| Terpineol | 0.05 |
| Eugenol | 0.01 |
| Thymol | 0.03 |
| water | Rest |
| Total | 100 |

The composition given above provides anti bacterial effect on skin within 15 seconds.

1. A method for providing an anti-microbial effect to skin comprising the steps of:
(a) applying to the skin a skin treatment composition comprising
(I) a polymer complex or mixture comprising
(i) polymer A selected from the group of polyacrylic acid and polyvinyl alcohol, and
(ii) a polymer B selected from the group of polyvinyl alcohol and polyethylene oxide; wherein Polymers A and B are not the same and
(II) an essential oil selected from thymol, terpineol, eugenol or mixture thereof; and
(b) waiting for at least 5 seconds.

2. A method according to claim 1, wherein polymer A is polyacrylic acid and polymer B is polyethylene oxide.

3. A method according to claim 1, wherein polymer A is polyacrylic acid and polymer B is polyvinyl alcohol.

4. A method according to claim 1, wherein polymer A is polyvinyl alcohol and polymer B is polyethylene oxide.

5. A method according to claim 1, wherein the composition is wiped or rinsed from the skin after step (b).

6. A method according to claim 1, wherein polymer A is present in a concentration of between 0.001 and 25% by weight of the composition.

7. A method according to claim 1, wherein polymer B is present in a concentration of between 0.001 and 20% by weight by weight of the composition.

8. A method according to claim 1, wherein the essential oil is present in a concentration of between 0.001 and 10% by weight of the composition.

9. A method according to claim 1, wherein the composition further comprises a second essential oil, preferably a second and a third essential oil.

10. A method according to claim 1, wherein the pH of the composition is between 2 and 9.

11. Use of a combination of
(a) a polymer complex or mixture comprising
(i) polymer A selected from the group of polyacrylic acid and polyvinyl alcohol, and
(ii) a polymer B selected from the group of polyvinyl alcohol and polyethylene oxide; wherein Polymers A and B are not the same and
(b) an essential oil selected from thymol, terpineol, eugenol or mixture thereof, for providing an anti-microbial effect on skin.

12. Use according to claim 11, wherein polymer A is polyacrylic acid and polymer B is polyethylene oxide.

13. Use according to claim 11, wherein polymer A is polyacrylic acid and polymer B is polyvinyl alcohol.

14. Use according to claim 11, wherein polymer A is polyvinyl alcohol and polymer B is polyethylene oxide.
